(19) United States
(12) Patent Application Publication
(10) Pub. No.: US 2015/0257664 A1
(43) Pub. Date: Sep. 17, 2015

(54) HEART MONITORING SENSOR

(71) Applicant: Avatar Engineering Corporation, Fountain Hills, AZ (US)

(72) Inventor: Anthony G. Esposito, Fountain Hills, AZ (US)

(21) Appl. No.: 14/658,936

(22) Filed: Mar. 16, 2015

Related U.S. Application Data
(60) Provisional application No. 61/954,548, filed on Mar. 17, 2014.

(51) Int. Cl. A61B 5/024

(52) U.S. Cl. CPC A61B 5/02444; A61B 5/02438; A61B 2562/166; A61B 2503/40

(57) ABSTRACT

A heart monitoring sensor having a printed circuit board with a depressed inner area, which, together with a flexible bulb extending from a perimeter for the depressed area, forms a sealed cavity. A sensor, such as a pressure sensor, is mounted within the sealed cavity. The bulb is releasably attached to the exterior of an animal near a heart pulse-detection point, and pressure changes within the cavity from the animal's heart pulse are sensed by the pressure sensor. The sensor output is provided outside the heart monitor by wired or wireless connection. In a particular embodiment, the sensor may be an active ranging sensor, and may range off of a reflective inner surface of the bulb. The active ranging sensor may be sonar, light reflective, (such as lidar), or Doppler. For a horse, the preferred location is in a natural depression underneath the horse's tail near the horse's dock.

### CROSS REFERENCE TO RELATED APPLICATIONS

This application claims the benefit of U.S. provisional patent application 61/954,548 filed Mar. 17, 2014 by the same inventor.

### FIELD OF ART

The present invention relates to a sensor for monitoring a heart rhythm, for example, of a horse. The present invention more closely relates to a equine heart rate sensor attachable under a horse tail near the horses dock.

### STATEMENT REGARDING FEDERALLY SPONSORED RESEARCH AND DEVELOPMENT

None.

### SUMMARY OF THE INVENTION

A pressure transducer within a sealed cavity at least partially bounded by an elastic and resilient bulb shaped to be secured proximate a vein or artery near the skin surface, for example, under the tail of a horse near the dock to sense blood pressure variations in the caudal vein or artery.

### DESCRIPTION OF THE FIGURES OF THE DRAWINGS

The present invention will hereinafter be described in conjunction with the following drawing figures, wherein like numerals denote like elements, and

FIG. 1 is a left side elevation view illustrating an exemplary embodiment of a heart monitoring sensor installed on a horse, according to a preferred embodiment of the present invention;

FIG. 2 is a side elevation cross-sectional view illustrating an exemplary embodiment of the heart monitoring sensor discussed above in regard to FIG. 1, according to a preferred embodiment of the present invention;

FIG. 3 is a top plan view illustrating an exemplary embodiment of the heart monitoring sensor of FIG. 2, according to a preferred embodiment of the present invention;

FIG. 4 is a diagrammatic view illustrating an exemplary embodiment of the heart monitoring sensor of FIG. 2, according to a preferred embodiment of the present invention;

FIG. 5 is a top plan view illustrating a second exemplary embodiment of the heart monitoring sensor, according to a preferred embodiment of the present invention;

FIG. 6 is a side elevation view illustrating the exemplary embodiment of the heart monitoring sensor of FIG. 1, according to a preferred embodiment of the present invention;

FIG. 7 is a side elevation view illustrating another exemplary embodiment of the heart monitoring sensor, according to a preferred embodiment of the present invention; and

FIG. 8 is a side elevation view illustrating yet another exemplary embodiment of the heart monitoring sensor, according to a preferred embodiment of the present invention.

### DETAILED DESCRIPTION OF THE INVENTION

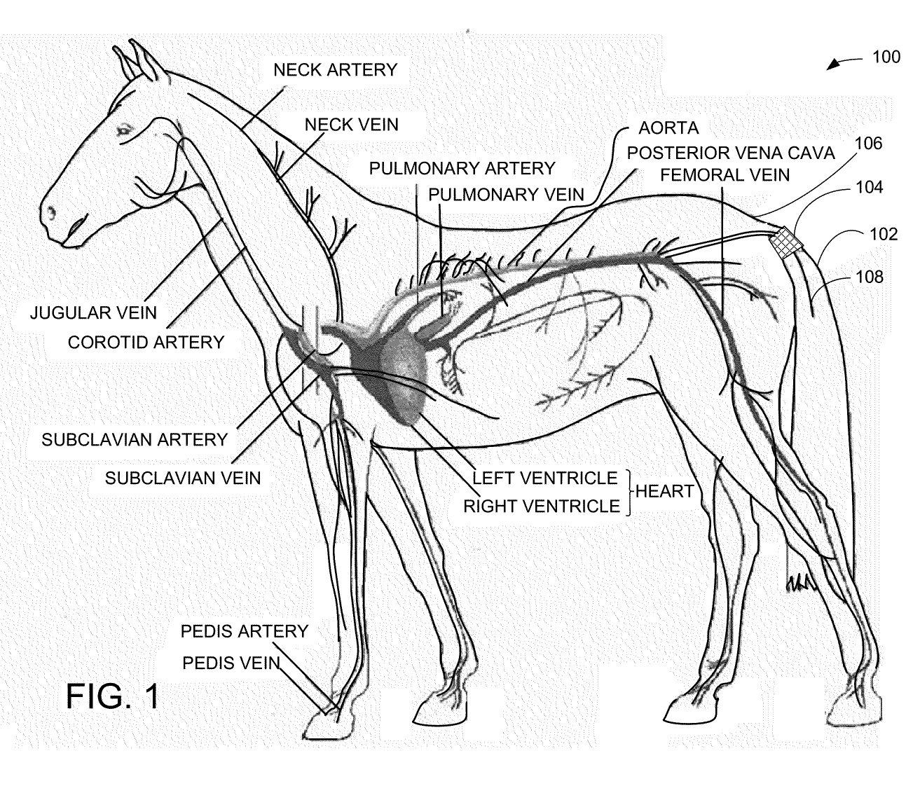

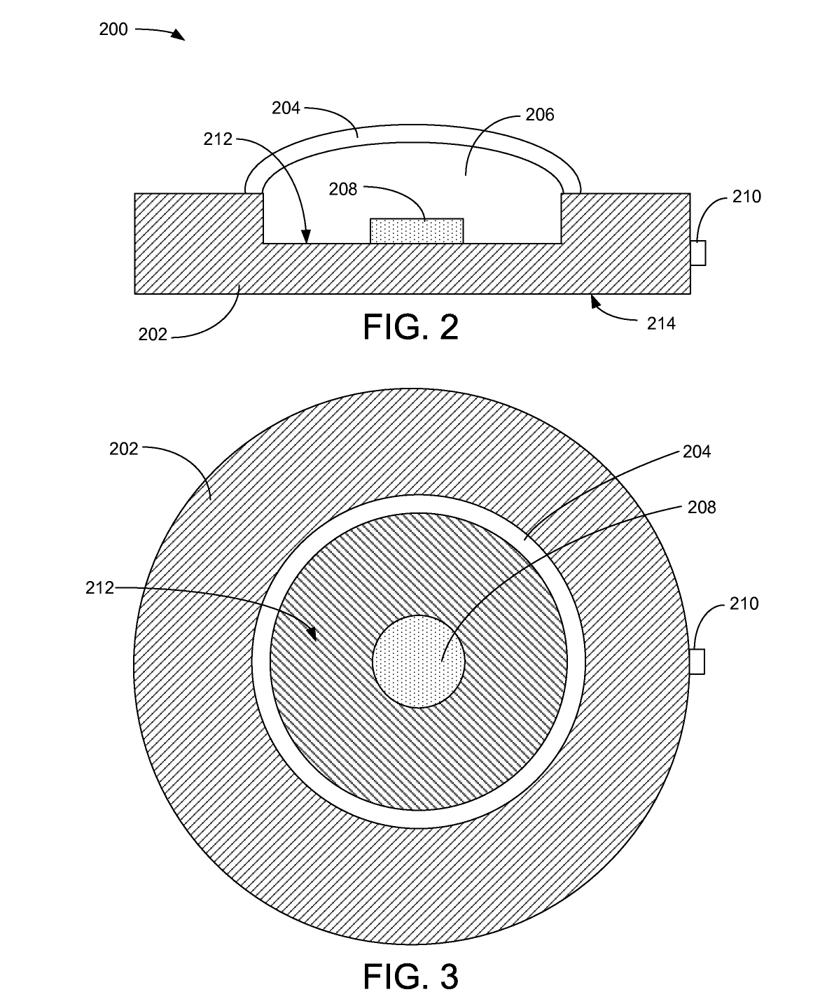

FIG. 1 is a left side elevation view illustrating an exemplary embodiment of a heart monitoring sensor 200 (see FIG. 2) installed on a horse 100, according to a preferred embodiment of the present invention. Visible in this view is a wrap 104 for securing the heart monitoring sensor 200 to the underside of the tail 102 of the horse 100 near the horse's dock 106 to sense the pressure in the caudal vein or artery 108. This positioning of the sensor reduces any distractions to the horse 100 as is common with neck-mounted sensors or difficulty in remaining connected, as is common with leg-mounted sensors. The uses and adaptations of the present invention are not limited to horses, which are used herein as an example, as the device can be useful with any mammal that has a heartbeat and a vein or artery near enough to the skin to allow a pulse to be detected.

FIG. 2 is a side elevation cross-sectional view illustrating an exemplary embodiment of the heart monitoring sensor 200 discussed above in regard to FIG. 1, according to a preferred embodiment of the present invention. Printed circuit board (PCB) 202 has a lower, or depressed inner surface 212 that is covered by a flexible and resilient bulb 204 that extends from the PCB 202 to a point above the PCB 202 to form sealed cavity 206. Bulb 204 may be seated and sealed on top of PCB 202, as shown, or seated and sealed on depressed inner surface 212: either perimeter will serve. Sealed cavity 206 contains, for example, a pressure transducer 208 that produces a signal which can be accessed at connector 210. In exemplary operation, bulb 204 is secured against the underside of the horse's tail 102 such that pressure in the caudal vein or artery 108 compresses or releases pressure on bulb 204, causing pressure transducer 208 to change the pressure-dependent signal it generates. The signals are processed into data elsewhere, and the data is used to evaluate the health of the horse 100.

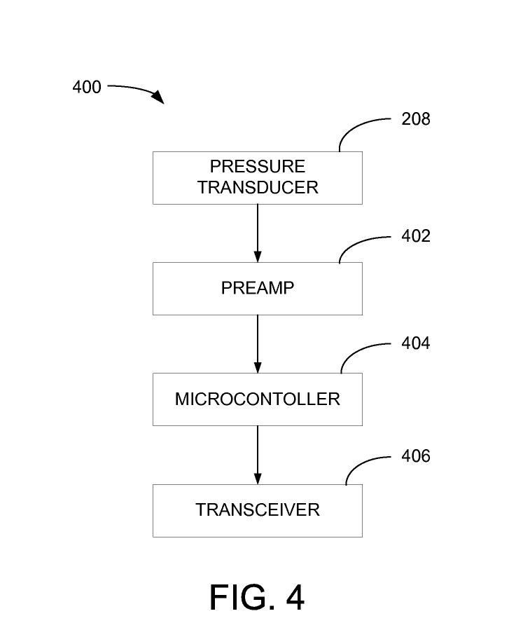

In various embodiments, connector 210 may be located at various locations on the external surfaces of PCB 202. The signal pathway through the PCB may be through an interplane connection and/or a plated though hole. In a particular embodiment, connector 210 may be omitted, and the signals may be transmitted wirelessly from inside the sealed cavity 206 using additional electronics (see FIG. 4). In another particular embodiment, the connector 210 may be a wireless antenna.

FIG. 3 is a top plan view illustrating an exemplary embodiment of the heart monitoring sensor 200 of FIG. 2, according to a preferred embodiment of the present invention. Bulb 204 is shown as transparent beyond the sealed edges. The relative size and shape of the pressure transducer 208 is not a limitation of the invention. Small size and weight is preferred for the entire heart monitoring sensor 200.

FIG. 4 is a diagrammatic view illustrating an exemplary embodiment of the heart monitoring sensor 200 of FIG. 2, according to a preferred embodiment of the present invention. Additional electronics that may be mounted on the PCB 202 in addition to pressure transducer 208 include a preamplifier 402 for amplifying the signals produced by the pressure transducer 208, a microcontroller 404 to control, for example, sampling rate and to, for further example, format signals for transmission, and a transceiver 406 for transmitting sampled signals to a data processing facility and for receiving instructions for the microcontroller 404. The additional electronics 402, 404, and 406 may be mounted within the cavity 206 or on the external surface of PCB 202. The listed additional electronics 402, 404, and 406 are merely exemplary and does not preclude other or additional electronics on the heart monitoring sensor 200.

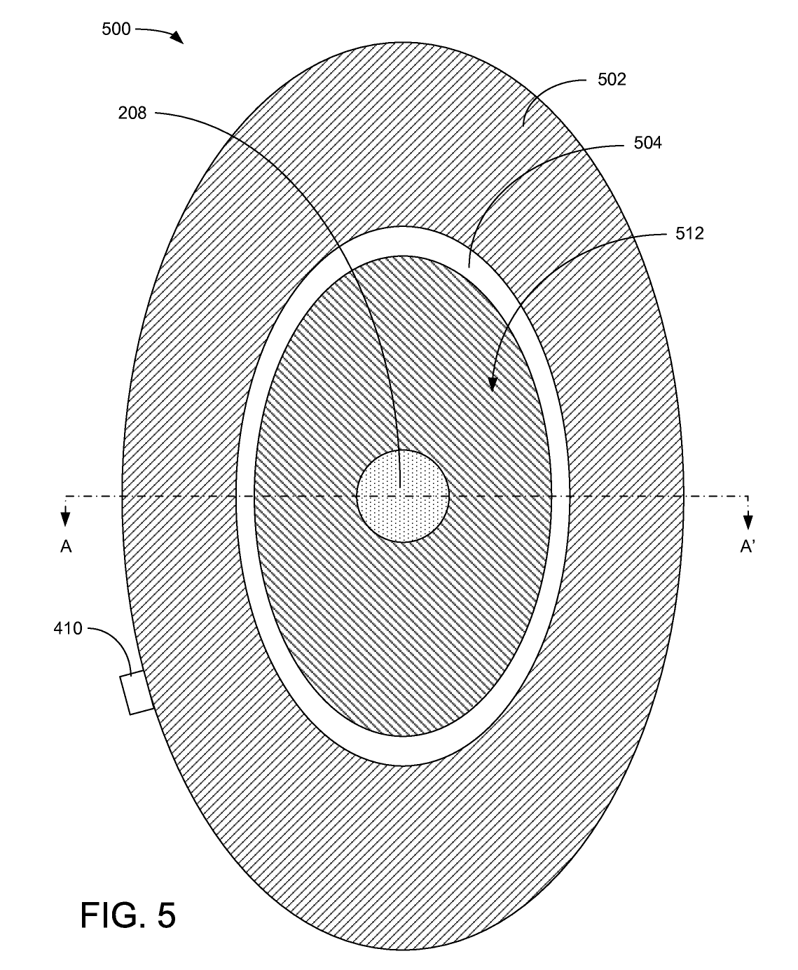

FIG. 5 is a top plan view illustrating a second exemplary embodiment of the heart monitoring sensor 500, according to a preferred embodiment of the present invention. Heart monitoring sensor 500 illustrates that the shape of heart monitoring sensor 200 is not a limitation of the invention. The oval heart monitoring sensor 500 uses a PCB 502 with a lowered, or depresses, inner surface 512 that is covered by bulb 504 to make a sealed cavity over pressure transducer 208. Connector 410 has the same scope as with heart monitoring sensor 200. A cross section AA′ would look like FIG. 1, except for the placement of connector 410.

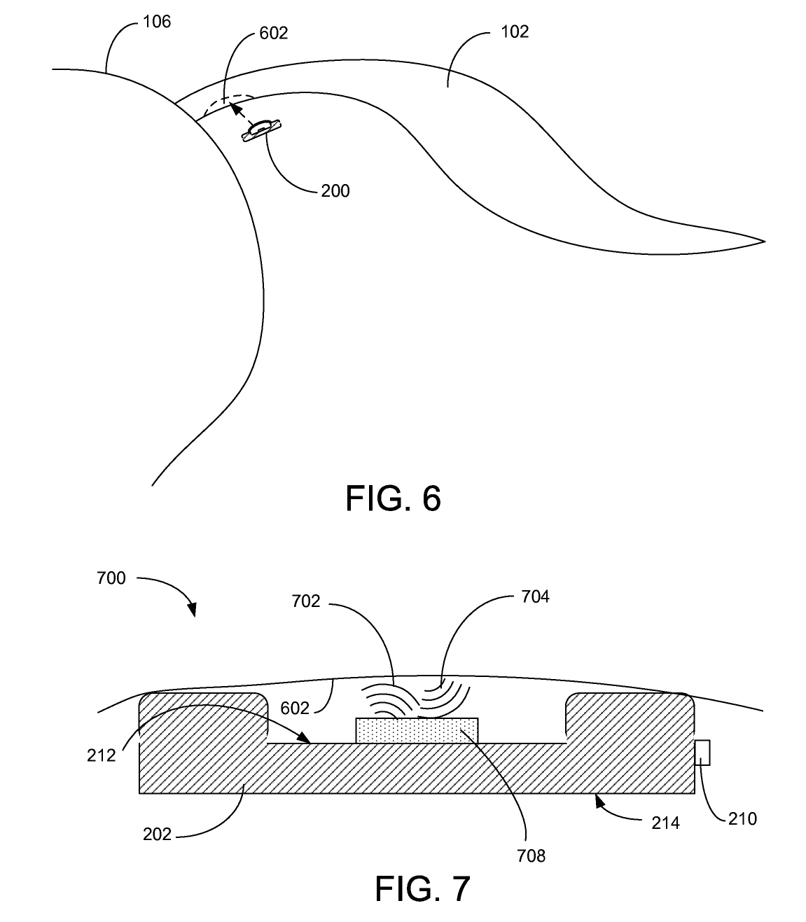

FIG. 6 is a side elevation view illustrating the exemplary embodiment of the heart monitoring sensor of FIG. 1, according to a preferred embodiment of the present invention. As can be seen in FIG. 6, the tail 102 has a natural depression 602 on the underside of the tail 102 and shaping the heart monitoring sensor 200 or 500 to fit conformally in this depression is preferred. In some embodiments, a conformal shape designed to fit horses 100 generally is within the scope of the present invention. In another embodiment, the shape may be customized to fit a particular horse 100 or other creature. Various sizes, adapted to various sizes or breeds of horses, are also within the scope of the invention, as a heart monitoring sensor 200 for a Clydesdale may not be a good fit for an American Quarter Horse. Once emplaced in the depression 602, the heart monitoring sensor 200 or 500 may be secured by wrap 104, or similarly effective means.

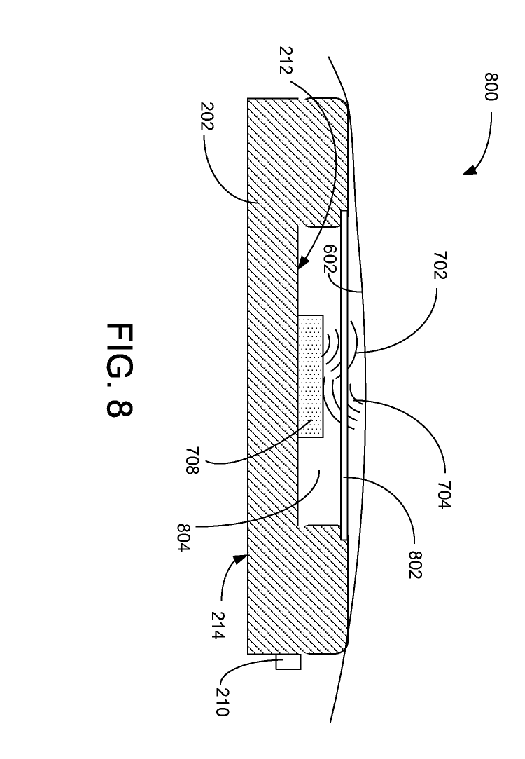

FIG. 7 is a side elevation view illustrating another exemplary embodiment of the heart monitoring sensor 700, according to a preferred embodiment of the present invention. In yet another embodiment, the heart monitoring sensor may be optical or sonic and may use or omit the bulb 204. In this illustrated optical embodiment, an optical sensor (transceiver 708) measures pressure changes as the changes over time in the range from transceiver 706 to the surface of the horse 100 in cavity 602. The frequency of the output signal 702 and the reflected signal 704 is naturally much higher than the frequency of the pulse of horse 100. Consequently, good resolution of the pulse rate may be obtained. In a particular embodiment, the Doppler signature of the pulse may be exploited for remote vibration sensing.

FIG. 8 is a side elevation view illustrating yet another exemplary embodiment of the heart monitoring sensor 800, according to a preferred embodiment of the present invention. In this illustrated optical embodiment, a transparent membrane 802 may be used over cavity 804 to prevent contamination of the transceiver 708. Membrane 802 may be rigid or flexible.

In yet another optical embodiment, the bulb 204 is used with an additional reflective surface on the underside of the bulb 204. The distance between transceiver 708 and the underside of bulb 204 is repeatedly measured over time to determine the pulse rate. Again, the Doppler signature of the horse pulse may be exploited for remote vibration sensing.

In sonic embodiments, similar to the optic embodiment of FIG. 7, the collection of data regarding range changes over time are found by sound ranging. Preferably, the frequency of the air vibrations are above 25 KHz, to prevent distracting the horse 100 or the rider. As with the optical embodiments, the frequency of the sound is high compared to the frequency of a horse's pulse, and so good resolution can be obtained. The Doppler signature of the horse pulse may be exploited for remote vibration sensing. Sound ranging to the underside of the bulb 204 is also within the scope of the present invention, and has the advantage of reducing the risk of contamination of the transceiver.

I claim:

1. A heart sensor comprising,
a. a printed circuit board having a depressed portion;
b. a flexible bulb extending from a perimeter of said depressed portion to a distance above said printed circuit board, wherein said bulb and said depressed portion form a sealed cavity;
c. a sensor within said sealed cavity; and
d. a sensor signal pathway from said sensor within said cavity to at least one point outside said cavity.

2. The heart sensor of claim 1, further comprising a preamplifier and a microcontroller mounted on said printed circuit board within said cavity.

3. The heart sensor of claim 1, further comprising a device for securing said heart sensor to the body of a living creature.

4. The heart sensor of claim 1, further comprising a water-resistant covering for said printed circuit board.

5. The heart sensor of claim 1, wherein said bulb comprises a reflective inner surface.

6. The heart sensor of claim 1, wherein said sensor comprises one of a pressure transducer and a remote vibration sensor.

7. The heart sensor of claim 6, wherein said ranging sensor determines range from said sensor to a reflective inner surface of said bulb.

8. The heart sensor of claim 6, wherein said ranging sensor is at least one of a sonar ranging sensor and a light-ranging sensor.

9. The heart sensor of claim 1, wherein said sensor signal pathway comprises a wireless signal pathway.

10. The heart sensor of claim 1, wherein said sensor signal pathway comprises one of an inter-plane connection and a through-hole connection on said printed circuit board.

11. The heart sensor of claim 1, wherein the combination of said printed circuit board and said bulb comprise a shape conformal to a portion of a living creature's body.

12. The heart sensor of claim 11, wherein a combination of said printed circuit board and said bulb comprise a shape conformal to a natural depression on the underside of a horse's tail.

13. The heart sensor of claim 12, further comprising a device for releasably attaching at least said combination of said printed circuit board and said bulb to said horse's tail.

14. A heart sensor comprising,
a. a printed circuit board having a depressed portion;
b. a flexible bulb extending from a perimeter of said depressed portion to a distance above said printed circuit board, wherein said bulb and said depressed portion form a sealed cavity;
c. a sensor within said sealed cavity;
d. a sensor signal pathway from said sensor within said cavity to at least one point outside said cavity;
e. a preamplifier mounted on said printed circuit board within said cavity;
f. a microcontroller mounted on said printed circuit board within said cavity; and
g. a device for securing said heart sensor to the body of a living creature.

15. The heart sensor of claim 14, wherein said bulb comprises a reflective inner surface.

16. The heart sensor of claim 14, wherein said sensor comprises at least one of a pressure transducer and a ranging sensor.

17. The heart sensor of claim 14, wherein a combination of said printed circuit board and said bulb comprise a shape conformal to a natural depression on the underside of a horse's tail.

18. A heart sensor comprising,
a. a printed circuit board having a depressed portion;
b. a flexible bulb extending from a perimeter of said depressed portion to a distance above said printed circuit board, wherein said bulb and said depressed portion form a sealed cavity;
c. a sensor within said sealed cavity;
d. a sensor signal pathway from said sensor within said cavity to at least one point outside said cavity;
e. a preamplifier mounted on said printed circuit board within said cavity;
f. a microcontroller mounted on said printed circuit board within said cavity; and
g. a device for securing said heart sensor to the body of a living creature.

19. The heart sensor of claim 18, wherein:
a. said bulb comprises a reflective inner surface;
b. said sensor comprises at least one of a pressure transducer and a ranging sensor;
c. wherein said ranging sensor determines range from said sensor to said reflective inner surface of said bulb; and
d. said ranging sensor is at least one of a sonar ranging sensor and a light-ranging sensor.

20. The heart sensor of claim 18, wherein the combination of said printed circuit board and said bulb comprise a shape conformal to a portion of a living creature's body.

21. A heart sensor comprising,
a. a printed circuit board (PCB) having a depressed portion;
b. a sensor within said depressed portion;
c. wherein said sensor is operable to detect a pulse when said PCB is abutted against an animal's skin in a position where a vein or artery is close to the surface of the skin;
d. a sensor signal pathway from said sensor within said cavity to at least one point outside said cavity.

22. The heart sensor of claim 21, wherein said sensor comprises a pressure transducer.

23. The heart sensor of claim 21, wherein said sensor comprises a remote vibration sensor.

24. The heart sensor of claim 23, wherein said remote vibration sensor comprises one of and optical sensor and a sonic sensor.
